(19) United States
(12) Patent Application Publication
(10) Pub. No.: US 2025/0352267 A1
(43) Pub. Date: Nov. 20, 2025

(54) EMBEDDED LASER FIBER FOR ASPIRATED STONE ABLATION

(71) Applicant: GYRUS ACMI, INC. D/B/A OLYMPUS SURGICAL TECHNOLOGIES AMERICA, Westborough, MA (US)

(72) Inventor: Kurt G. Shelton, Bedford, MA (US)

(21) Appl. No.: 19/280,854

(22) Filed: Jul. 25, 2025

Related U.S. Application Data
(63) Continuation-in-part of application No. 18/048,010, filed on Oct. 19, 2022, now abandoned.
(60) Provisional application No. 63/270,797, filed on Oct. 22, 2021.

(51) Int. Cl. A61B 18/26; A61B 1/018; A61B 1/05; A61B 1/06; A61B 18/00

(52) U.S. Cl. CPC A61B 18/26; A61B 1/018; A61B 1/05; A61B 1/0676; A61B 2018/00982

(57) ABSTRACT

A single-use or reusable cap or an objective head to a scope can be used for integrating a laser fiber within the objective head but outside a working channel of the scope. The endoscope maintains an attached or embedded permanent laser fiber to ablate particulates that are suctioned into the endoscope assembly via a suction conduit. With such integration, there is no need to manipulate the laser fiber into a working channel; rather, a practitioner, e.g., physician, can move the medical device, including the laser fiber, as an entire unit into the patient toward the target stone and allow for suction of the stone through a working channel without blocking of the working channel by the laser fiber.

### CROSS-REFERENCE TO RELATED APPLICATIONS

This application is a continuation-in-part application of U.S. patent application Ser. No. 18/048,010, filed on Oct. 19, 2022, which claims priority to and the benefit of the filing of U.S. Provisional Patent Application Ser. No. 63/270,797, filed on Oct. 22, 2021. The specifications, claims, and figures of all of the applications cited above are hereby incorporated by reference herein in their entirety.

### TECHNICAL FIELD

The exemplary and non-limiting embodiments described herein are related to medical devices. More specifically, the exemplary and non-limiting present disclosure relates to techniques, products, assemblies, methods, and/or apparatuses for a medical device and mechanism for the use of a laser fiber outside of a working channel of a scope device.

### BACKGROUND

Medical examination tools generally referred to as scopes, such as ureteroscope, cystoscope, nephroscope, gastroscope, endoscope, and the like, can be used to inspect the inside of a body for the purpose of diagnosing and curing abnormalities.

For example, an endoscope has a distal end comprising an optical or electronic imaging system and a proximal end with controls such as for manipulating the device or for viewing the image. An elongated shaft connects the proximal and distal ends. Some endoscopes allow a physician to pass one or more tools down a working channel, for example, to pass an instrument to resect tissue or retrieve objects.

Over the past several decades, several advances have been made in the field of endoscopy, and in particular relating to the methods of insertion of instruments into the endoscope for break up and retrieval of physiological calculi, e.g., calcification or concretion of material, in the body. Different techniques have been developed in the medical field to perform endoscopic procedures, which include different sized instruments, such as the instruments used in ultrasonic or other acoustic lithotripsy, pneumatic lithotripsy, electro-hydraulic lithotripsy, and laser lithotripsy. For example, laser lithotripsy can include breaking up of calculi using a green light or holmium laser. Generally, a laser fiber is inserted into a working channel of an endoscope toward the target, which may interfere with the use of the working channel for other instruments or purposes, and which may therefore involve instrument swapping, a longer procedure, or the like.

As the medical professional inserts the laser fiber into the endoscope, the insertion of the laser fiber involves precision and time in order to reach the distal end of the medical device, which can be a meter in length. During this time, the patient is generally partially or fully sedated.

### BRIEF DESCRIPTION OF THE DRAWINGS

The present disclosure will be apparent from the following more particular description of examples of embodiments of the technology, as illustrated in the accompanying drawings. The drawings are not necessarily to scale, emphasis instead being placed upon illustrating embodiments of the present disclosure. In the drawings, like numerals may describe similar components in different views. Like numerals having different letter suffixes may represent different instances of similar components. The drawings illustrate generally, by way of example, but not by way of limitation, various embodiments discussed in the present document.

Various embodiments in accordance with the present disclosure will be described with reference to the drawings, in which:

FIG. 1A illustrates an example schematic diagram of a scope objective head with a built-in fiber;

FIG. 1B illustrates an example schematic diagram of an objective head with an attachable laser fiber;

FIG. 2A illustrates an example schematic diagram of an endoscope objective head with a built-in fiber;

FIG. 2B illustrates an example schematic diagram of an endoscope objective head with an attachable laser fiber;

FIG. 3 illustrates an example schematic diagram of an endoscope objective head with a built-in fiber;

FIG. 4A illustrates an example perspective view of an objective head;

FIG. 4B illustrates an example perspective view of an objective head;

FIG. 5 illustrates an example schematic diagram of an attachable objective head with a built-in fiber;

FIG. 6 illustrates an example perspective view of an attachable endoscope objective head with a built-in fiber;

FIG. 7 illustrates a flow chart showing a method of applying using an objective head that can be utilized;

FIG. 8 a flow chart showing a process of applying an attachable objective head to a reusable scope that can be utilized; and

FIG. 9 is a block diagram illustrating exemplary components of a sample endoscope.

### DETAILED DESCRIPTION

An example of an endoscope assembly of the present disclosure can include an endoscope apparatus that is intended to be used in the monitoring, diagnosing, and/or treating of a patient such as to alleviate an injury or disease. The endoscope apparatus can include or use an energy delivery component or assembly, such as for transferring energy or delivering energy to the tissue. The energy delivery component or assembly can include a laser fiber component of the medical device, which can deliver electromagnetic energy to a target such as to break a calculus, stone, or other tissue or other target into smaller pieces such as to permit removal of the pieces from the patient or from a portion of the patient's body.

However, the act of breaking the tissue and removing tissue from a body cavity can involve using multiple pieces of equipment in a working or other channel of the medical device that may be only 1-4 millimeters in lateral dimension. The equipment to be placed within a channel of the endoscope may include an irrigation supply, a suction conduit, an image/video camera(s), illumination lighting mechanisms, laser fiber(s), or the like. The use of multiple pieces of equipment in such a confined space can cause over-crowding or may involve a time-consuming sequence of inserting and removing various pieces of equipment into or from the working channel to provide such equipment with access to the target.

This disclosure can help provide solutions to the problem created by confined space in a working channel, such as by permitting use of an embedded laser fiber outside of the working channel. For example, the laser fiber(s) can be configured to extend proximally at least partially in a separate sheath that can be maintained outside of the normal working channel of the endoscope. This can help provide for additional space in the working channel, while creating a maintained and consistent space for a distal tip of the laser fiber(s) to be placed adjacent to the working channel. This can help enable a medical provider to save room in the working channel of the apparatus for additional or different equipment, to save time in the procedure by not having to place the laser fiber into the working channel only to have to swap it out for providing irrigation and/or suction or another piece of equipment, and to maintain consistency in the location of the laser fiber in relation to the endoscope. This disclosure can help provide easier use of the laser fiber with an endoscope, such as by providing for an attached or embedded laser fiber within, on, or near a reusable or disposable endoscope.

In some examples, relatively expensive components, such as a camera, a light source, various electronic components, and others, may be considered to be portions of an endoscope, and may therefore be reusable. In some examples, the relatively inexpensive components, such as the working channel, the laser fiber, and others, may be a separate unit or module that can snapped onto the endoscope, and may therefore be single-use.

In some examples, a laser fiber can be embedded in a snap-on piece that can be clipped onto an endoscope. The endoscope can have a reusable part, such as the reusable part of an objective head that includes the light source and imaging sensor, which can be more expensive than other components. The endoscopic system can include a single-use part, separate from the endoscope and attachable to the endoscope, such as by snapping onto the endoscope. The single-use part can include the working channel and an integrated laser fiber attached along a bottom of the working channel, which can be less expensive than other components, such as the light source and the imaging sensor. A practitioner can clip the single-use piece, includes the working channel and the laser fiber, onto the reusable objective head of the endoscope.

In other examples, the endoscope can have a more typical design that includes the working channel and has an objective head that includes the light source and imaging sensor. The snap-on piece can allow the laser fiber to be clipped onto the tip of the endoscope. The laser fiber may run along the entire length of the endoscope inside the outer jacket of the endoscope. In some examples, the factory assembly of the components can run the laser fiber along the length of the endoscope. In some examples, the factory assembly can glue and run the laser fiber beside the endoscope. Using attachable components can add slightly to a diameter of the endoscope, by an amount roughly equal to a thickness of the laser fiber.

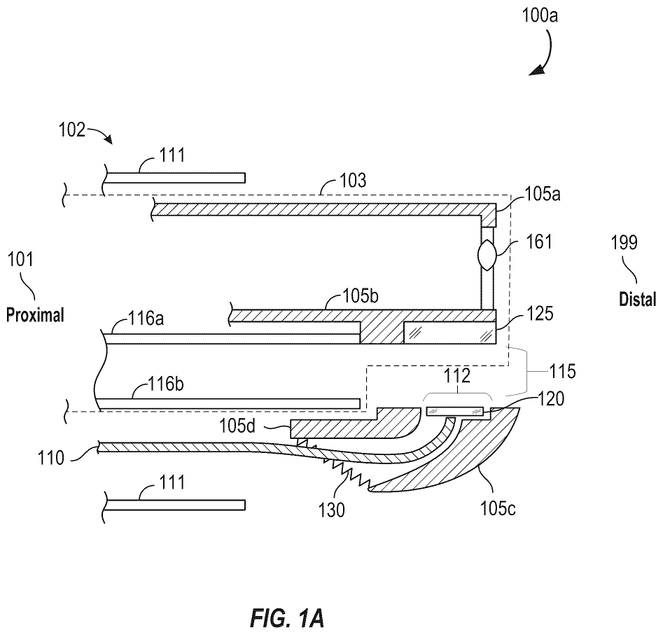

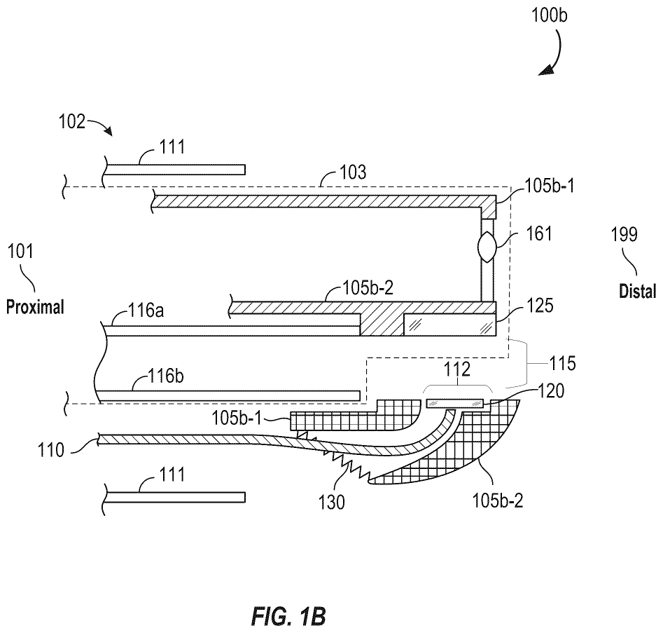

FIG. 1A illustrates a partial cross-sectional view 100a of an example of a portion of an endoscope assembly 102 that includes an endoscope 103. The cross-sectional view 100a shows a proximal portion 101 and a distal portion 199 of the endoscope assembly 102. An objective head 105 (a-d) of the endoscope assembly 102 may be configured such as shown in FIG. 1A. For example, the objective head 105 (a-d) may be configured to include one or more engagement features, such as to snap-on or otherwise be attached about a distal portion 199 of the endoscope assembly 102. An endoscopic procedure may include imaging or otherwise visualizing sections of the inside of a body, performing biopsies, performing surgeries, and/or performing ablations of body tissue and removing a portion of the body tissue from the patient's body via a working channel 115 of the endoscope assembly 102. One or more elements of the objective head 105 (a-d), such as elements 105c and 105d, may be configured to be user-attachable and user-detachable to the distal portion 199 of the endoscope 103 in the endoscope assembly 102. The objective head 105 (a-d) may include one or more of illumination or visualization optics, such as an objective lens for a camera or a focal plane array (FPA) or other imaging device, an illumination fiber or fiber bundle, or the like.

The endoscope assembly 102 can include the working channel 115. The working channel 115 can be defined as a tubular or other lumen, such as between an upper wall 116a and a lower wall 116b. The working channel 115 can provide a lumen that can provide an open space in the endoscope assembly 102, such as to enable passage of one or more instruments such as can be used for direct or indirect visual inspection, diagnosis, or treatment of hollow spaces, body cavities, or lumen (e.g., an opening inside a tubular body structure that is lined by body tissue) during the endoscopic procedure.

For example, for laser lithotripsy, the medical device or endoscope assembly 102 can include a laser fiber that is desired to have its distal tip located toward the working channel opening 115 at the distal side 199. The distal side 199 of the working channel opening 115 can further include an additional second opening or second channel 112. For example, the additional second opening 112 can be defined by an upper portion 105(d) of the objective head 105 and a lower portion 105(c) of the objective head 105, which can provide for a second channel that can be located below the working channel 115 defined by walls 116(a) and wall 116(b). The objective head 105 can include or be constructed out of plastic or stainless steel, or other suitable material. The second opening 112 can allow for a second channel outside of the working channel 115 that can provide a pathway outside of the working channel 115, such as for passing an embedded or attached laser fiber(s) 110, which can also include passage via a sheath 111, such as a direct pathway for the laser fiber(s) 110.

The laser fiber(s) 110 may include one or more laser fibers such as can be used in laser lithotripsy, such as can include breaking up of calculi using a green light, YAG, holmium laser, or other suitable laser or electromagnetic or other ablation energy source. Reference to the laser fiber 110 in the singular should be understood to extend to a bundle of multiple laser fibers.

As shown in FIG. 1A, the laser fiber 110 is attached through or embedded in the separate channel 112 outside of the working channel 115. This can allow for an increase in space in the working channel 115 for use as an irrigation or suction conduit, or insertion of one or more separate fluid conduits into the working channel such as to permit irrigation, suction, or both. In FIG. 1A, at least a distal end of the laser fiber 110 can be attached in place within the separate channel 112, such as using an adhesive 130, a mechanical fastener or fixture, another fixation component, or any combination of these.

In FIG. 1A, the separate channel 112 for the laser fiber 110 can include a window 120. The window 120 can help separate a distal end of the laser fiber 110 in the channel 112 from the working channel 115 or the target calculi stone or other tissue to be ablated via laser lithotripsy energy emitted from the distal end of the laser fiber 110. The window 120 may be substantially transparent to the wavelength of electromagnetic laser lithotripsy energy emitted from the distal end of the laser fiber 110. The window 120 may include a silica glass or sapphire or other window such as to help protect against burn-back from the laser fiber lithotripsy energy. The window 120 can help inhibit or prevent fragments, dust, or small pieces of the calculi being suctioned into the distal end 199 of the working channel 115. Such fragments can be suctioned toward the proximal end 101 of the working channel 115 and do not enter into the separate second channel 112. The window 120 may help prevent the distal tip of the laser fiber 110 from directly coming into contact with the target calculi to be ablated, such as via laser lithotripsy. The window 120 can also help enables the laser fiber 110 to maintain a desired gap between a distal tip of the laser fiber 110 and the calculi or other target. This can further help avoid the distal tip of the laser fiber 110 being affected by any burn-back from ablating the target.

The working channel 115 can permit a medical professional, such as a doctor, surgeon, veterinarian, or engineer, to insert additional ancillary medical equipment into the working channel 115 of the endoscope assembly 102, such as to assist with the particular procedure being performed. For example, such ancillary medical equipment may include a thermometer, an imaging system, a camera, an infusion pump, a stone retrieval basket, a stent deployment catheter, or other ancillary device that may be introduced inside the working channel 115 of the endoscope assembly 102 and advanced toward the target at the distal end 199 of the scope. The working channel 115 may include one or more additional ports, such as toward the proximal end 101 of the scope, such as to allow insertion of one or more ancillary devices into the working channel 115 of the scope. The distal objective head 105 may include one or more objective lenses or optics or other optical interface 161. The optical interface 161 can be optically transparent at the desired illumination, imaging, or visualization light wavelengths. Thus, the optical interface 161 can help allow illumination light to be delivered therethrough, or for imaging or visualization to be carried out using the optical interface 161. The laser fiber 110 can thus be embedded or otherwise located in the separate channel 112, which can help avoid the laser fiber 110 being located in the working channel 115. This, in turn, can help avoid the laser fiber 110 blocking access to other ancillary instruments, or to irrigation to or aspiration of fragments or fluid from the target location. In this way, the laser fiber 110 does not float within the working channel 115 causing blockage in removing calculi or calculi fragments via the working channel. Also, the laser fiber 110 does not have to be removed in order to allow access to the target by ancillary instruments inserted through the working channel 115. By avoiding the need to swap the laser fiber 110 in and out for other ancillary instrumentation to be used within the working channel 115, the procedure can be made much easier and faster.

In FIG. 1A, the endoscope assembly 102 can further include a heat shield 125. The heat shield 125 can be located upon and interconnected with the objective head 105(b), such as in an optical pathway of laser lithotripsy electromagnetic energy being emitted from the distal tip of the laser fiber 110, such as through the window 120. The heat shield 125 can be made of a material that is capable of withstanding such incident laser lithotripsy energy, such as to help protect against overheating of any portions of the distal objective head 105 at are located beyond the heat shield 125 or any medical equipment or components carried by the distal objective head 105, such as the objective lens or other optical interface 161. For example, the heat shield 125 can be an optical diffuser or a heat-sinking shield 125 that can be configured to protect optical components of the endoscope assembly 102, such as those carried by the distal objective head 105 beyond the heat shield 125. For example, the heat shield 125 can include a reflective surface that can include a material or structure capable of reflecting the wavelengths associated with incident electromagnetic energy from the laser fiber 110 away from the distal objective head 105 or away from electronic or optical components carried by the distal objective head 105 and located beyond the heat shield 125. In this way, the reflective surface of the heat shield 125 will not absorb a lot of heat but will instead reflect it away thereby scatter the heat from the laser fiber 110. For example, the heat shield 125 can include a mirror-like coating such that laser light that hits the heat shield 125 can be reflected away and not cause burn-back. A diffractive or other scattering surface can additionally or alternatively be used for the heat shield 125.

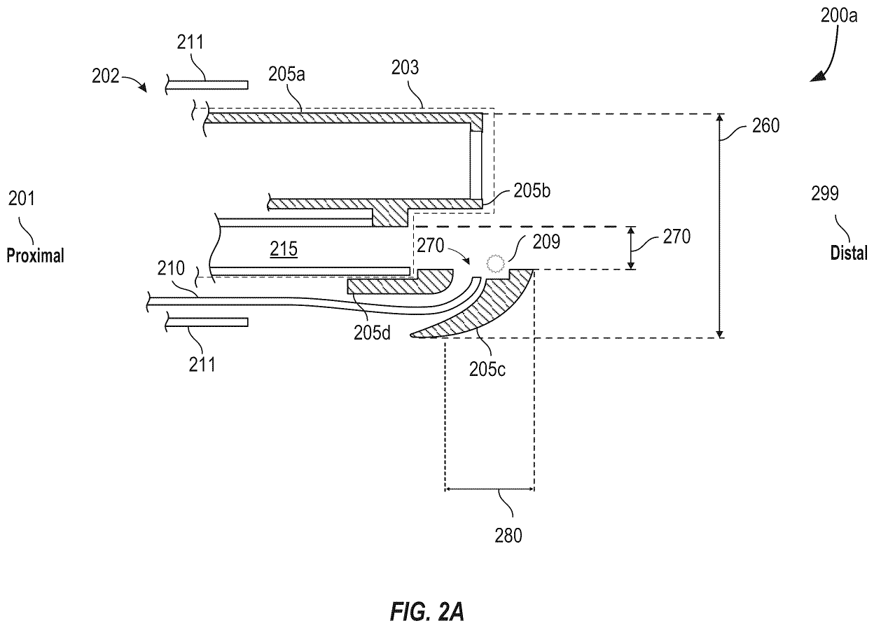

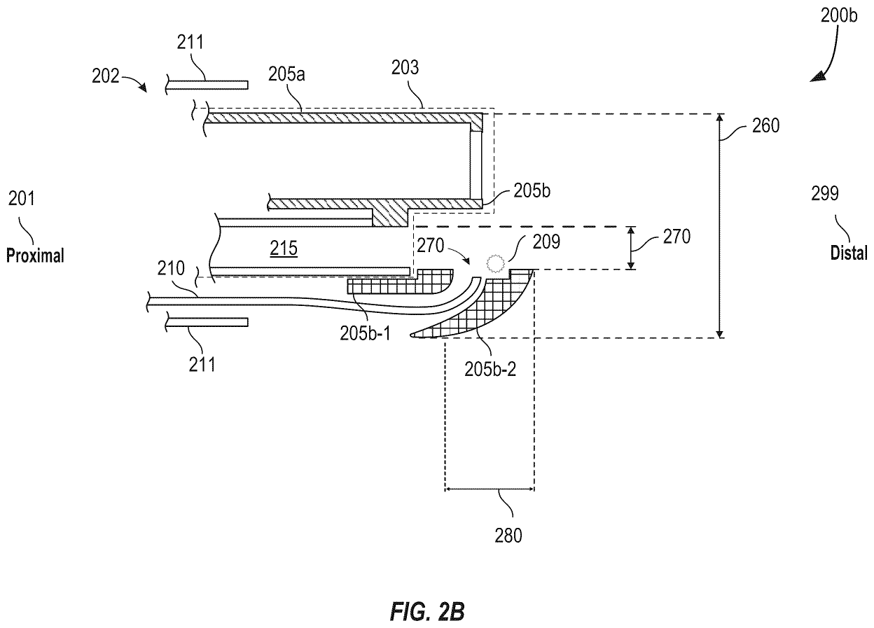

FIG. 1B illustrates an example schematic diagram 100b of a reusable endoscope objective head 105b-1 and 105b-2 that can be attachable and detachable by an end-user to a single-use or reusable endoscope. In FIG. 1B, the scope can include the lower assembly 150b-1 and 150b-2 that incorporates a separate laser fiber pathway for placement of the laser fiber 110. FIG. 1B is similar in many respects to FIG. 1A, sharing certain similar features and components. For example, the re-usable objective head 105b-1 and 105b-2 may correspond to the upper portion of the objective head 105a-b in relation to FIG. 1A. In FIG. 1B, the reusable objective head 105b-1 and 105b-2 may incorporate the lighting mechanisms, camera, or optical components 161 in the upper portion of the objective head. However, the lower assembly 150b-1 and 150b-2 can be included as part of the single-use or re-usable scope, to which the re-usable objective head 105b-1 and 105b-2 can be attached and detached by the end-user. The lower assembly 150b-1 and 150b-2 may optionally be end-user attachable and detachable, or simply may be an integrated part of the single-use or re-usable scope. FIG. 2A illustrates an example of a schematic diagram 200a of portions of an endoscope assembly 202 with an endoscope objective head 205(a-b) with a built-in laser fiber 210, similar to that explained above with respect to FIG. 1A. FIG. 2A depicts an illustrative example of available spacing and sizing to provide additional context relating to the small available space for performing procedures.

In FIG. 2A, a lower half of the objective head 205(c-d) is shown as protruding closer toward the distal end 299 of the endoscope assembly 202, including an endoscope 203, than an upper half of the objective head 205(a-b). The entire objective head 205(a-d) may only be a few millimeters in size, such as three to four millimeters in height 260. Such a protrusion 280 can allow for the distal tip of the laser fiber 210 to be inserted such that it emits laser energy at an angle that is not incident upon the upper half of the objective head 205(a-b). Instead, the laser light that exits the distal tip of the laser fiber can be incident upon the target calculi at a location that is more distal than the upper half of the objective head 205(a-b). This can help avoid heating effects upon the upper half of the objective head 205(a-b) or upon electronic or optical components carried or housed by the upper half of the objective head 205(a-b). This can help reduce or avoid the need to include a heat shield 125, as explained above with respect to FIG. 1A, in which the lower half of the objective head was recessed from the upper half, rather than protruding beyond.

The amount of protrusion 280 of the lower half of the objective head 205(c-d) can optionally be specified or adjusted based on one or more characteristics of a calculi 209 being removed. For example, in order to treat larger calculi, such as stones of a diameter (or similar cross-sectional size dimension) greater than three millimeters, the angle between the distal portion of the laser fiber 210 and a central longitudinal axis defined by the working channel 215 can be shallower than for treating a stone of a size smaller than one to two millimeters.

The laser fiber 210 can be used to alone or in combination with one of a suction conduit or an irrigation conduit, which can be catheters located in the working channel 215, or which can be provided via separate channels in the endoscope assembly 202. For example, laser lithotripsy energy from the laser fiber 210 can be delivered toward calculi 209 while the calculi 209 is being suctioned toward the distal end 299 of the endoscope assembly 202, such as toward a distal opening of the working channel 215 through which suction is being applied to aspirate one or more resulting fragments of the calculi 209 generated by the laser lithotripsy. At the desired angle of insertion 270 of the laser fiber 210, the calculi 209 can be broken into smaller pieces, such as to be removed through a suction conduit placed in the working channel 215 or through the working channel 215 itself, when suction is applied thereto at the proximal end 201 of the endoscope assembly 202. The lateral cross-sectional size 270 of the distal opening of the working channel 215 may be only millimeters in width, such as one to two millimeters. Therefore, it can be desirable to apply the laser lithotripsy to break down the size of the calculi 209 into pieces that are smaller than the size of the working channel 215 such as to permit such fragments to be aspirated from the distal end 299, without causing a clog, tear, or the like.

The calculi 209 can be brought toward the distal end 299 of the endoscope assembly 202 (or vice versa). Irrigation and suction can be used to help accomplish this. For example, the calculi can be brought toward the distal end 299 of the endoscope assembly 202 via an irrigation and/or suction ebb and flow, such as can be created by an irrigation conduit that can be placed within the working channel 215. One or more other techniques can additionally or alternatively be employed to help break up calculi, including ultrasonic or another acoustic lithotripsy, optoacoustic lithotripsy, pneumatic lithotripsy, electro-hydraulic lithotripsy (EHL), etc.

FIG. 2B illustrates an example schematic diagram 200b of a reusable endoscope objective head 205a and 205b. As explained above with respect to FIG. 1A, the lower assembly 250b-1 and 250b-2, which incorporates a separate laser fiber pathway 235 for placement of the laser fiber 210, may be included as features of the single-use or re-usable endoscope 203, and need not be end-user attachable or end-user detachable from the endoscope 203, unless so desired.

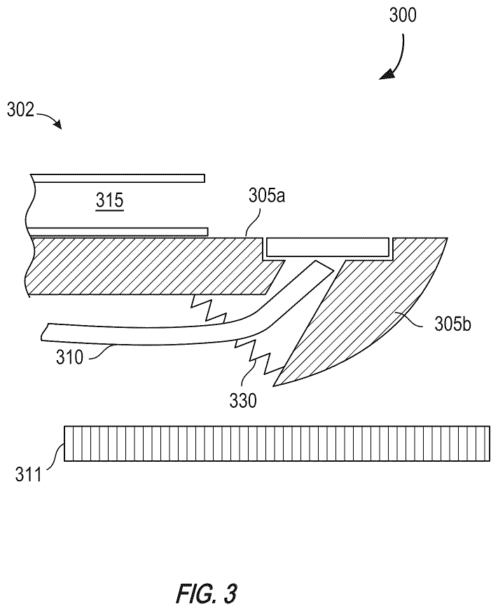

FIG. 3 illustrates an example of a schematic diagram 300 showing a lower portion of an objective head 303 with a built-in or attached laser fiber 310. FIG. 3 is similar in many respects to FIGS. 1A, 1B, 2A, and 2B, sharing certain similar features and components. For brevity, only specific elements are detailed and described with reference to FIG. 3.

In FIG. 3, a sheath 311 can be provided as an endoscope access sheath 311, into the which a distal portion of an endoscope assembly 302 can be inserted into the body of a patient. As a user, such as a doctor or surgeon, introduces the endoscope into the sheath 311, the laser fiber 310 can be pulled along within the access sheath 311 together with the push of the endoscope through the sheath 311. In other words, there is no need for the user to separately or distinctly push or fit the laser fiber 310 through the working channel 315 of the endoscope after the endoscope is placed into the sheath 311. Instead, the laser fiber 310 is introduced together with the endoscope from the beginning as the endoscope is introduced into the sheath 311 with the distal end of the laser fiber 310 being attached to the distal objective head 305, which, in turn, can have been already attached to a distal end of the endoscope.

The access sheath 311 is not required. Where an endoscope is not introduced via an access sheath 311, such as in a case in which a scope is being inserted directly into a patient's ureter without any access sheath 311, the laser fiber 310 is still conveniently pulled along while inserting the endoscope, since the distal end of the laser fiber 310 is attached to the distal objective head 305, which, in turn, is attached to a distal end of the endoscope. Therefore, even without such access sheath 311, the laser fiber 310 does not require separate insertion. Not using an access sheath 311 can help where it is desirable to perform the procedure with the least amount of ureteral distension and associated discomfort.

Without the access sheath 311, the laser fiber 310 can still maintained in position using an adhesive 330 or other fixation of a distal end of the laser fiber 310 to the distal objective head 305 of the scope. The adhesive 330 may include a gel that is optically transparent at the wavelength of the laser that is coupled to the laser fiber 310. For example, at a wavelength of 1940 nanometers, in adhesive can be transparent to permit passage of laser lithotripsy electromagnetic energy in a wavelength range between 1900 and 2160 nanometers. Other adhesives for wavelength ranges may be used for other treatments.

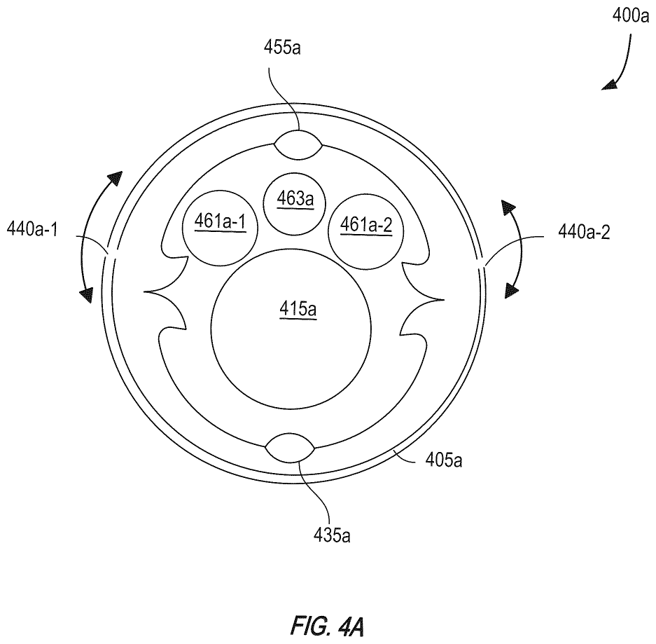

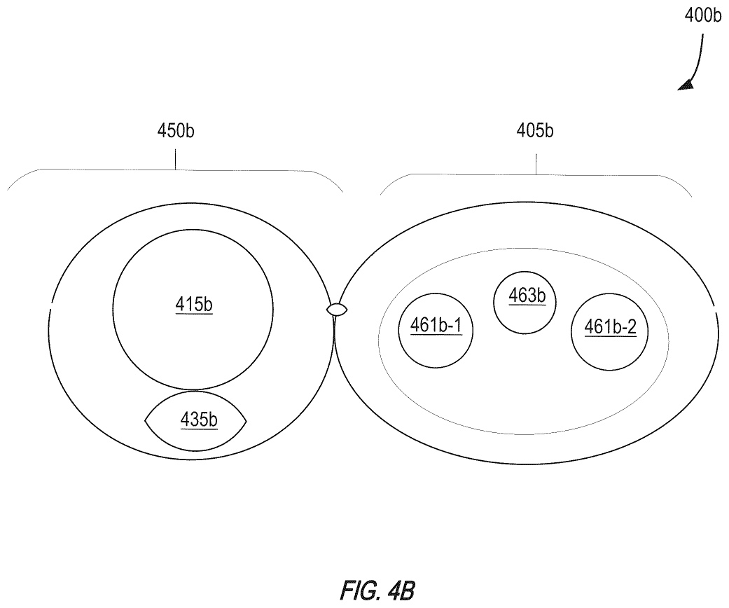

FIG. 4A conceptually illustrates an example of a perspective view 400a looking toward a distal end of an objective head 405a that is attached to a distal end of an endoscope assembly 102, 202, or the like.

The objective head 405a may include a reusable cap, such as which can be operatively coupled with one or more laser fiber(s) that can be set into a laser fiber pathway 435a of the objective head 405a that is located outside of the working channel 415a of the scope. A reusable objective head 405a can be attached to a distal end of a reusable scope. The reusable objective head 405a can permit at least a distal end of the laser fiber(s) to be preset into an embedded channel in the objective head 405a that is located outside of, away from, or otherwise different from a working channel 415a of the endoscope assembly.

The objective head 405a may include a built-in embedded pathway 435a for integrating or embedding one or more laser fibers through the pathway into the endoscope assembly towards the proximal end of the endoscope. More than one embedded pathway 435a may be included in the same objective head 405a, if desired, such as a second embedded pathway 455a. The embedded pathway(s) can be configured to provide for a separate channel or channels, outside a working channel 415a, such as in which a corresponding laser fiber or other elongate component can be placed.

The objective head 405a may be attached as a cap to a distal portion of an endoscope assembly, such as in a snap-on manner, or in a threaded or other rotational engagement or other fastening manner that can be twisted in either a clockwise 440a-1 or counterclockwise 440a-2 direction maintaining a smooth configuration between the objective head 405a and the distal portion of the endoscope assembly. Such a smooth configuration can help avoid any crevices or sharp areas of the entire apparatus that might otherwise be snagged inside the body cavity or within an endoscope sheath.

In FIG. 4A, the objective head 405a can include one or more additional channels, separate from the working channel 415a and separate from the embedded laser fiber pathway 435a. Such one or more separate additional channels may be used for introducing or placing additional equipment or providing additional functionality through such one or more other conduits or channels. For example, the objective head cap 405a may include a left optical interface or other port 461a-1 and a right optical interface or other port 461a-2, such as can be respectively accessed via corresponding separate and independent channels, such as for illumination, imaging or other visualization, irrigation, suction, or other auxiliary functionality as desired for a procedure being performed. The left and right optical interfaces 461a-1 and 461a-2 may include one or any combination of one or more lenses, one or more waveguides, one or more light sources, or other optical or photonic elements such as for delivering illumination light, receiving an imaging or visualization optical signal, among other things. For example, multiple illumination light sources may be used such as to help maintain uniform illumination across a field of view at or near a desired target. A dedicated port or optical interface 461a-1 can be used to provide illumination lighting, such as to help assist user visualization or imaging of the target via the scope. One or more additional ports may be included in the objective head 405a, such as an imaging or visualization port 463a that may include optics or otherwise provide a specified channel for visualization camera or imaging equipment for use in the endoscopic procedure.

FIG. 4B conceptually illustrates an example of a perspective view 400b looking toward a distal end of an objective head 405b that can be configured to be laterally end-user attachable to and detachable from the endoscope assembly 102, 202, such as in the side-by-side manner shown in FIG. 4B, rather than an objective head being placed over and about a distal end of the endoscope assembly 102, 202, or the like, such as described and explained above with respect to FIG. 4A.

In FIG. 4B, the objective head 405b may be a reusable objective head that may include a reusable housing that can carry or contain one or more lighting mechanisms 461b-1 and 461b-2 and a camera or camera assembly 463b, which may be reusable components of the reusable objective head. The objective head 405b can further be operatively coupled with a disposable or single-use or other scope assembly 450b that can include one or more laser fiber(s) that can be set into a laser fiber pathway 435b of the single-use assembly 450b, such as outside of the working channel 415b of the scope. For example, the laser fiber pathway 435b can be located laterally adjacent to a portion of the working channel 415b of the scope. A reusable objective head 405b can be attached to a distal end of the disposable or re-usable scope, such as by an end-user, using a snap-on or other engagement technique, such as described elsewhere herein. For example, the reusable objective head 405b can be operatively coupled laterally in a side-by-side manner to a distal portion of the single-use assembly 450b, such as to permit at least a distal end of the laser fiber(s) to be preset into an embedded channel in the scope assembly 450b. For example, the reusable objective head 405b can be connected with or attached to the disposable or single-use or other scope assembly 450b by a clip, attachment component, screw-in attachment, or the like, such as described elsewhere herein. Further, the reusable objective head 405b can be attached with or connected to the disposable or single-use or other scope assembly 450b in a lateral position corresponding to the appropriate attachment points.

Additionally, the reusable objective head 405a-b (including the one or more illumination lighting devices 461b-1 and 461b-2 and camera assembly 463b) can optionally be integrated with the scope already (as opposed to an attachable/detachable component from the scope) and the objective head 405a-b and endoscope integration can include reusable components. For example, a laser fiber(s) can be integrated proximate to but outside of the working channel 415b, such as at an integration location 435b, where the laser fiber is integrated with the scope such that both can be used together such as in a single-use disposable manner. The laser fiber integration channel 435b may be located in a predetermined location around or near the working channel 415b, may be moved to different locations around or near the working channel 415b, or may be molded or otherwise embedded in an area proximate to the working channel. The laser fiber and working channel 415b can be assembled and/or disassembled with the objective head 405b, such as laterally in a side-by-side manner, such as using the snap-on or other affixation mechanisms described herein.

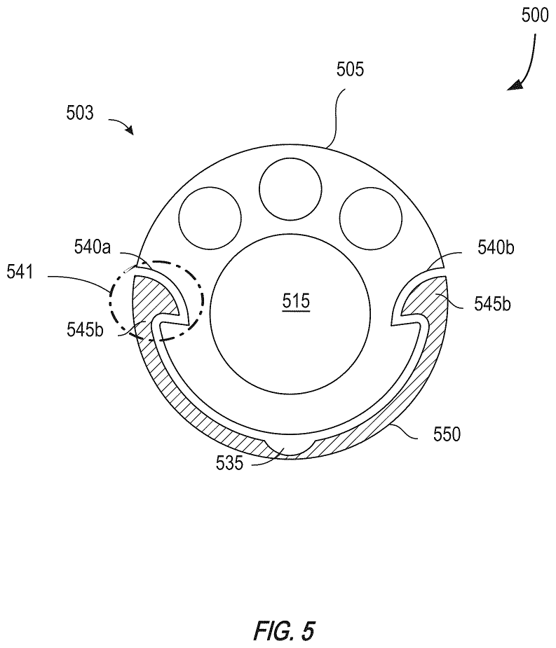

FIG. 5 conceptually illustrates a perspective view 500 looking toward the distal end of an endoscope assembly 503 including an objective head 505 that can include an attachable cap 550. The objective head 505 may include one or more receiving or other engagement features 545a and 545b, such as can respectively be located on the opposing left and right side of the objective head 505 respectively. The engagement features 545a and 545b may be used to affix the objective head 505 to a distal portion of the endoscope assembly 503, such as through engagement with mating or interlocking corresponding engagement features 540a and 540b that can be located on a distal portion of the scope.

The engagement features 545a and 545b can include a snap-on or snap-in feature to engage with the corresponding engagement features 540a and 540b on the scope. The resilience of the objective head 505 or a clip portion thereof can provide a biasing spring force such as to permit snap-fit or snap-on engagement, disengagement, or both, such as by a physician or other end-user. The snap-in locking mechanism 541 formed by the connection of the engagement feature 545a and interlocking attachment piece 540a can be configured to robust enough such as to maintain a snapped-in or locked position despite the forces placed on the snap-in locking mechanism 541 by the endoscopic procedure, the torsion or rotational forces placed on the mechanism by the end-user of the endoscope assembly during the endoscopic procedure, and/or the forces placed on the locking mechanism 541 from internal pressure of the patient.

The attachable cap 550 can attach the endoscope objective head 505 to the distal end of the endoscope. The objective head 505 or the cap 550 can include a built-in laser fiber pathway 535, such as which can carry a laser fiber that may be operably interconnected or operatively coupled to the objective head 505 outside of (e.g., below or downward from) a working channel 515 of the endoscope. A laser fiber pathway 535 may be additionally or alternatively placed laterally or above the working channel 515. The one or more laser fiber pathways 535 can be rotated by the end-user, such as in either a clockwise or counter-clockwise direction, such as within 360-degrees around the objective head.

Additionally, the objective head 505 and the working channel 515 can be integrated with or attached to the endoscope. As such, the objective head 505, which includes one or more illumination lighting devices, camera assemblies, or additional components, can be integrated in an endoscope that can include a working channel 515. The snap-on piece 550 that may be attached to the endoscope assembly can include a port or channel entrance 535, such as which can allow for the laser fiber to be clipped onto the endoscope assembly, such as described herein. The endoscope can be a reusable scope or a disposable or one-time use scope. The snap-on piece 550 can be a reusable piece or a disposable or one-time use piece, and the laser fiber can be a reusable fiber or a disposable or one-time use fiber. Other combinations of reusable or disposable components are possible, such as a disposable snap-on piece 550, with a disposable laser fiber, and a disposable endoscope or a mix of reusable and disposable components.

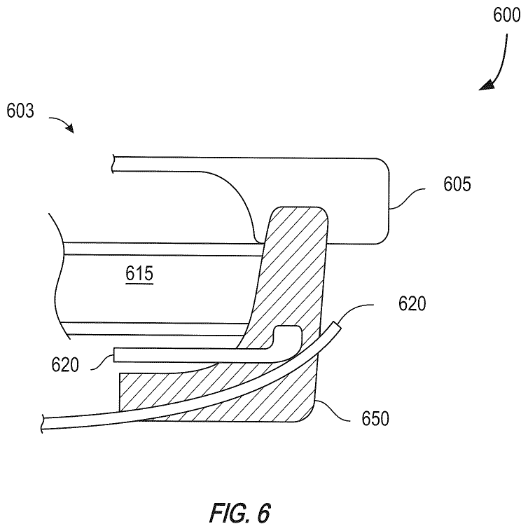

FIG. 6 illustrates an example of a perspective schematic diagram 600 of an attachable cap 650, such as which can be attached to an objective head 605 or which can include the objective head 605 and which can be directly attached to a distal portion of the endoscope. The attachable cap 650 can include a built-in pathway for a laser fiber 620.

The attachable cap 650 may be a single-use piece of equipment that is attachable by an end-user directly or indirectly to a single-use or re-usable endoscope assembly 603 before inserting a distal portion of the endoscope assembly into a patient. The attachable cap 650 can include an entrenched pathway for attaching the laser fiber 620 to the objective head 605 before insertion. This can help enable an end-user, such as a surgeon or medical professional, to insert the endoscope assembly together concurrently with the laser fiber 620.

Such an attachable cap 650 may be clipped to the objective head 605 (or directly to the endoscope) in a manner that creates an indentation or conduit or other pathway for the laser fiber 620 to extend back proximally out of the patient for connection to an external laser. As explained, the laser fiber pathway can be located outside of a working channel 615 of the endoscope assembly 603. The attachable cap 650 may further include a glass or other window 620, such as described with respect to FIG. 1A. The window 620 can be integrated into the attachable cap 650, such as to permit the attachable cap 650 to use the window 620 maintain a shield of protection of the distal tip of the laser fiber from burn-back from the energy emitted from the laser fiber 620.

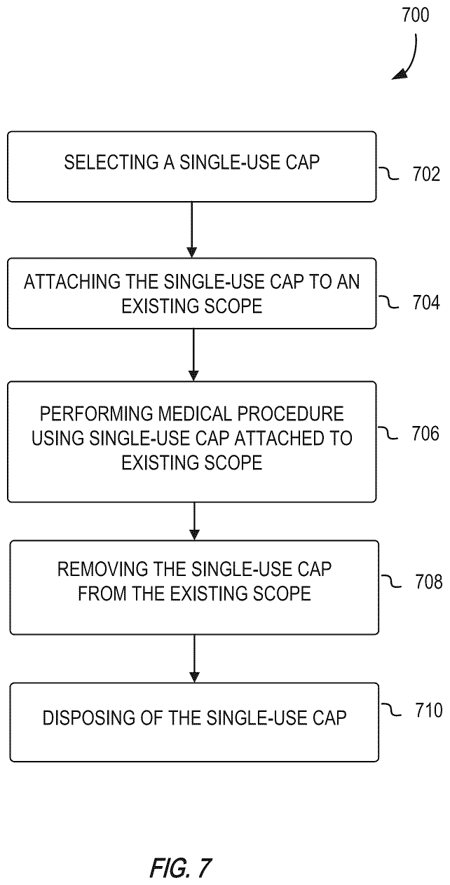

FIG. 7 shows an illustrative example of portions of a process 700 that may be used for applying an objective head integrated with a laser fiber and also integrated with a single-use endoscope. At operation 702, a single-use cap or a single use distal-objective head can be selected, such as for use with a single-use endoscope or a re-usable endoscope. At operation 704, the single-use cap or a single use distal-objective head can be attached to a single-use or re-usable scope. At operation 706, the end-user can perform the medical procedure using the single-use cap or single-use distal objective head having been attached to the existing single-use or re-usable endoscope. At operation 708, the single-use cap or the single-use distal objective head can be removed from the reusable endoscope, which can then be cleaned and sterilized, or the single-use cap or the single-use distal objective head can be disposed of after the procedure with the rest of the single-use endoscope. The removeable single-use cap may be received from a sterile packaging for the purpose of only being used in a single procedure.

The method or process 700 can help allow for customizing one or more components desired for performing the medical procedure. This can include providing or using one or more diagnostic tools, such as to be used to help identify the size, shape, material composition, and/or one or more additional characteristics of the calculi or target body to be removed from the cavity within the patient. Because one or more attributes of the calculi may be determined or discovered during the procedure, the process 700 can include allowing for interchanging one or more tools for an alternative one or more tools, such as can be based at least in part on one or more parameters of the target calculi.

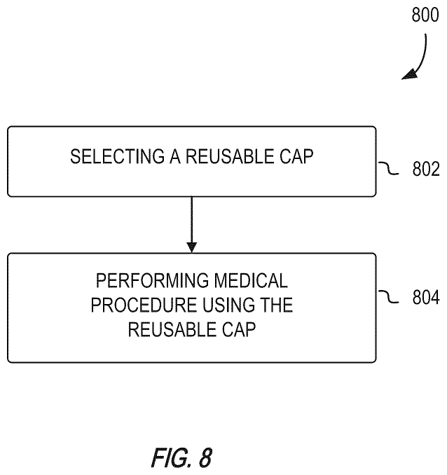

FIG. 8 is a flow chart showing an example of portions of a process 800 for applying an end-user attachable objective head to a reusable endoscope. The process 800 can include using a reusable or disposable cap or objective head 405a such as explained herein, including with respect to FIG. 4A. At operation 802, the process 800 can include selecting a reusable cap or distal objective head, such as which can provide a pathway for a laser lithotripsy fiber to extend from a distal end of the scope back proximally toward a proximal end of the scope, where the laser fiber can be connected to an external laser source. At operation 804, the process 800 can further include performing the medical procedure using the reusable cap or distal objective head being attached to a distal portion of the scope.

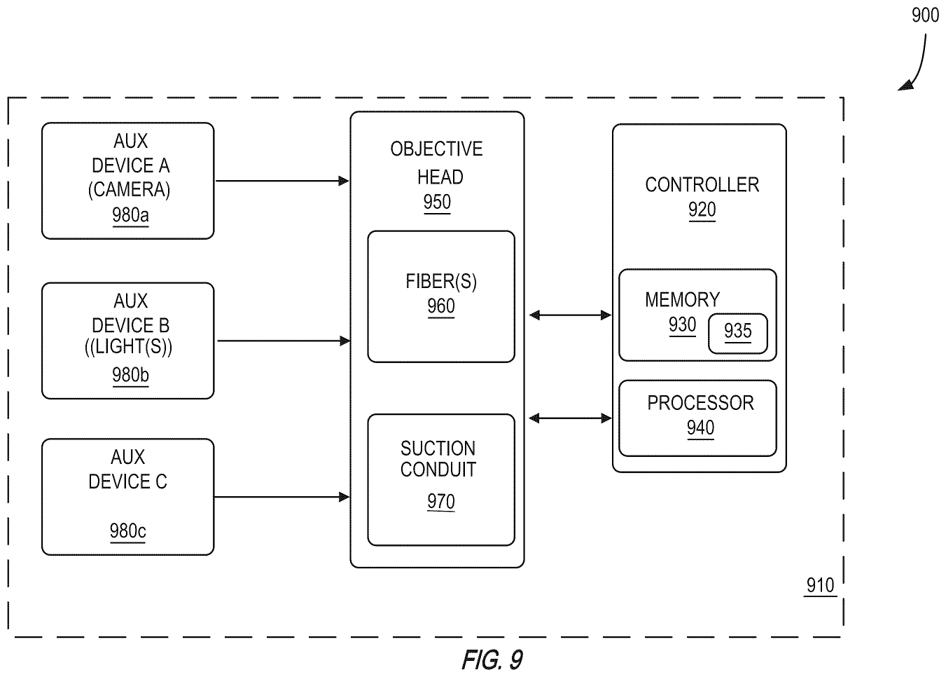

FIG. 9 depicts an example of block diagram of a system 900 illustrating components of a sample apparatus 910 that can include a laser portion that may be controlled by a controller 920. The controller 920 can include or be coupled to one or more memory 930 circuits, such as which can include stored instructions 935, and a processor 940. The controller 920 being operably interconnected to a memory 930, which may further be connected to non-transitory machine-readable medium instructions 935.

The controller 920 may be configured to coordinate an end-user issuing one or more laser pulses as desired to allow the medical professional to perform a medical operation. The fiber(s) 960 may include a laser fiber, such as the laser fiber 110 as depicted in FIG. 1A, which may be operably interconnected to the controller 920. The controller 920 may further be configured to receive information related to the feedback and transmission of the discharged laser fiber energy returned to the controller from the laser fiber(s) 960. The controller 920 may be configured to operated based on one or more pre-defined settings of the laser fiber(s) 910, where such pre-defined settings may include frequency, power settings, or the like.

The controller 920 can be connected to an objective head 950, such as to the objective head components 105a-d described herein. The objective head, the scope, or both can be used as a disposable or one-time-use apparatus or a reusable apparatus. The user may use the scope's imaging or visualization capabilities to observe a calculus relative to the apparatus 910 inserted into the patient.

The camera 980a may be used alone or in conjunction with one or more illumination lighting devices such as auxiliary device B 980b, which may be inserted through the scope via its working channel or via one or more separate dedicated channels that may be included, such as lighting channels 461a-1 and 461a-2 as described herein in connection with FIG. 4A. The one or more lighting devices may be operably interconnected with the camera or imaging devices, as well as any other components of the apparatus 910, such as the controller 920. The camera 980a and the light(s) 980b may further be configured in a single channel separated from the working channel or combined within one or more specified working channels. Additional auxiliary devices 980c may be operably interconnected to the apparatus 910 in order to provide for particular components or mechanisms needed or desired for a particular procedure or use of the scope.

The present technique may not require including or using all components. Also, the modules and/or devices discussed herein can be included or combined with any appropriate one or more devices or machines associated with an endoscopic procedure.

While examples described can include an embedded laser fiber(s) in an endoscope, such methods, devices, and techniques may similarly be provided for other medical devices used to provide scope technology with embedded laser(s) for use on humans, animals, or even non-living entities.

Each of these non-limiting examples can stand on its own or can be combined in various permutations or combinations with one or more of the other examples. The following examples detail certain aspects of the present subject matter to solve the challenges and provide the benefits discussed herein.

Example 1 can include an endoscopic apparatus for use for delivering laser lithotripsy energy to a target calculus within a body of a subject, via an elongate scope having a working channel, for fragmenting the target calculus into one or more fragments, the apparatus comprising: a distal objective head, sized and shaped to be coupled to a distal end of the scope; and a laser fiber, including a laser fiber distal end that is attached to the distal objective head at the distal end of the scope, the laser fiber extending proximally from the distal objective head outside the working channel of the scope toward a proximal end of the scope, wherein the laser fiber distal end is attached to the distal objective head at the distal end of the scope so as to be oriented toward a location beyond the working channel of the scope for delivering laser lithotripsy energy toward the target calculus being drawn toward the working channel of the scope via applied suction.

In Example 2, the subject matter of Example 1 optionally includes wherein the distal objective head is user-attachable and user-removable from the distal end of the scope.

In Example 3, the subject matter of Example 2 optionally includes wherein the distal objective head includes one or more engagement features adapted to engage with one or more corresponding mating features on the scope to at least one of user-attach or user-detach the distal objective head from the scope.

In Example 4, the subject matter of any one of Examples 1-3 optionally include the distal objective head is affixed at manufacture to the distal end of the scope.

In Example 5, the subject matter of any one of Examples 1-4 optionally include wherein the distal objective head also includes at least one of illumination optics, an imaging camera detector, or imaging optics.

In Example 6, the subject matter of Example 5 optionally includes wherein the laser fiber distal end is attached to the distal objective head at the distal end of the scope so as to be oriented toward a location beyond the working channel of the scope within a Field Of View of at least one of the imaging camera detector or imaging optics for observing the target calculus while delivering laser lithotripsy energy toward the target calculus being drawn toward the working channel of the scope via applied suction.

In Example 7, the subject matter of any one of Examples 1-6 optionally include wherein the laser fiber distal end is attached to the distal objective head at the distal end of the scope at a location that is less distal than a most distal end of the distal objective head.

In Example 8, the subject matter of any one of Examples 1-7 optionally include further comprising the scope.

In Example 9, the subject matter of any one of Examples 1-8 optionally include further comprising a window, separating a distal end of the laser fiber from the target calculus being drawn toward the working channel of the scope via applied suction, wherein the window is transmissible to electromagnetic energy received from the laser fiber.

In Example 10, the subject matter of any one of Examples 1-9 optionally include further comprising shield, between a distal end of the laser fiber and a shielded portion of the distal objective head, the shield configured to protect the shielded portion of the distal objective head from heat generated by electromagnetic energy emitted at the distal end of the laser fiber by at least one of reflecting electromagnetic energy away from the shielded portion of the distal objective head or sinking or otherwise dissipating heat from the electromagnetic energy away from the shielded portion of the distal objective head.

In Example 11, the subject matter of any one of Examples 1-10 optionally include further comprising an elongate scope sheath, including a central longitudinal sheath lumen sized and shaped to accommodate the scope inserted therein, wherein the laser fiber extends proximally from the distal objective head outside the working channel of the scope through the sheath lumen toward a proximal end of the scope.

In Example 12, the subject matter of any one of Examples 1-11 optionally include wherein a distal portion of the elongate scope is laterally end-user attachable to and detachable from the distal objective head in a side-by-side manner.

Example 13 is an endoscopic apparatus for use for delivering laser energy to a target within a body of a subject, the apparatus comprising: an elongate scope having a working channel; a laser fiber, including a laser fiber distal end that is attached at the distal end of the scope, the laser fiber extending proximally from the distal objective head outside the working channel of the scope toward a proximal end of the scope, wherein the laser fiber distal end is oriented toward a location beyond the working channel of the scope for delivering laser energy toward the target.

In Example 14, the subject matter of Example 13 optionally includes wherein the laser fiber is fixedly attached at manufacture to the distal end of the scope.

In Example 15, the subject matter of any one of Examples 13-14 optionally include wherein the laser fiber is user-attachable and user-removable from the distal end of the scope.

In Example 16, the subject matter of any one of Examples 13-15 optionally include wherein the laser fiber includes one or more engagement features adapted to engage with one or more corresponding mating features on the scope to at least one of user-attach or user-detach the laser fiber from the scope.

Example 17 is an endoscopic apparatus for use for delivering laser energy to a target within a body of a subject, via an elongate scope having a working channel, the apparatus comprising: a laser fiber, including a laser fiber distal end that is user-attachable to and user-attachable from the distal end of the scope, the laser fiber extending proximally from the distal objective head outside the working channel of the scope toward a proximal end of the scope, wherein when attached to the distal end of the scope, the laser fiber distal end is oriented toward a location beyond the working channel of the scope for delivering laser energy toward the target.

In Example 18, the subject matter of Example 17 optionally includes further comprising a distal objective head, sized and shaped to be coupled to a distal end of the scope, wherein the laser fiber is affixed to the distal end of the scope via the distal objective head.

In Example 19, the subject matter of Example 18 optionally includes wherein the distal objective head includes at least one of illumination optics, an imaging camera detector. or imaging optics.

In Example 20, the subject matter of any one of Examples 18-19 optionally include further comprising shield, between a distal end of the laser fiber and a shielded portion of the distal objective head, the shield configured to protect the shielded portion of the distal objective head from heat generated by electromagnetic energy emitted at the distal end of the laser fiber by at least one of reflecting electromagnetic energy away from the shielded portion of the distal objective head or sinking or otherwise dissipating heat from the electromagnetic energy away from the shielded portion of the distal objective head.

In Example 21, the subject matter of any one of Examples 17-20 optionally include further comprising a window, separating a distal end of the laser fiber from the target, wherein the window is transmissible to electromagnetic energy received from the laser fiber.

In Example 22, the subject matter of any one of Examples 17-21 optionally include further comprising an elongate scope sheath, including a central longitudinal sheath lumen sized and shaped to accommodate the scope inserted therein, wherein the laser fiber extends proximally from the distal end of the scope outside the working channel of the scope through the sheath lumen toward a proximal end of the scope.

In Example 23, the apparatuses or method of any one or any combination of Examples 1-22 can optionally be configured such that all elements or options recited are available to use or select from. Each of the non-limiting aspects or examples described herein may stand on its own or may be combined in various permutations or combinations with one or more of the other examples.

In the above description, various embodiments have been described. For purposes of explanation, specific configurations and details are set forth to provide a thorough understanding of the embodiments. However, embodiments may be practiced without allow the specific details. Furthermore, some features may be omitted or simplified in order not to obscure the embodiment being described.

The apparatus and methods recited in example embodiments of the present disclosure can apply to medical, veterinary, and/or engineering devices that are used to view cavities using a scope; this may include all the different variations of endoscopes, laparoscopes, ureteroscopes, colonoscope, arthroscope, etc. that fall within the general term “scope” as understood by a person having ordinary skill in the art. For ease of description and understanding, examples presented herein will refer to an “endoscope” as an example most generally understood in the field of lithotripsy; however, all such examples will not be recited, for brevity.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following aspects, the terms “including” and “comprising” are open-ended, that is, a system, device, article, composition, formulation, or process that includes elements in addition to those listed after such a term in an aspect are still deemed to fall within the scope of that aspect. Moreover, in the following aspects, the terms “first,” “second,” and “third,” etc. are used merely as labels, and are not intended to impose numerical requirements on their objects.

Method examples described herein may be machine or computer-implemented at least in part. Some examples may include a computer-readable medium, non-transitory computer-readable medium, or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact discs and digital video discs), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the aspects. Also, in the above Detailed Description, various features may be grouped together to streamline the disclosure. This should not be interpreted as intending that an unclaimed disclosed feature is essential to any aspect. Rather, inventive subject matter may lie in less than all features of a particular disclosed embodiment. Thus, the following aspects are hereby incorporated into the Detailed Description as examples or embodiments, with each aspect standing on its own as a separate embodiment, and it is contemplated that such embodiments may be combined with each other in various combinations or permutations. The scope of the invention should be determined with reference to the appended aspects, along with the full scope of equivalents to which such aspects are entitled.

What is claimed is:

1. An endoscopic apparatus for use for delivering laser lithotripsy energy to a target calculus within a body of a subject, via an elongate scope having a working channel, for fragmenting the target calculus into one or more fragments, the endoscopic apparatus comprising:
a distal objective head, sized and shaped to be coupled to a distal end of the elongate scope; and
a laser fiber, including a laser fiber distal end that is attached to the distal objective head at the distal end of the elongate scope, the laser fiber extending proximally from the distal objective head outside the working channel of the elongate scope toward a proximal end of the elongate scope, wherein the laser fiber distal end is attached to the distal objective head at the distal end of the elongate scope so as to be oriented toward a location beyond the working channel of the elongate scope for delivering the laser lithotripsy energy toward the target calculus being drawn toward the working channel of the elongate scope via applied suction, wherein the laser fiber includes one or more engagement features configured to engage with one or more corresponding mating features on the elongate scope to at least one of user-attach the laser fiber directly to the elongate scope or user-detach the laser fiber from the elongate scope.

2. The endoscopic apparatus of claim 1, wherein the distal objective head is user-attachable and user-removable from the distal end of the elongate scope.

3. The endoscopic apparatus of claim 2, wherein the distal objective head includes one or more engagement features adapted to engage with one or more corresponding mating features on the elongate scope to at least one of user-attach or user-detach the distal objective head from the elongate scope.

4. The endoscopic apparatus of claim 1, wherein the distal objective head is affixed at manufacture to the distal end of the elongate scope.

5. The endoscopic apparatus of claim 1, wherein the distal objective head also includes at least one of illumination optics, an imaging camera detector, or imaging optics.

6. The endoscopic apparatus of claim 5, wherein the laser fiber distal end is attached to the distal objective head at the distal end of the elongate scope so as to be oriented toward a location beyond the working channel of the elongate scope within a field of view of at least one of the imaging camera detector or imaging optics for observing the target calculus while delivering laser lithotripsy energy toward the target calculus being drawn toward the working channel of the elongate scope via applied suction.

7. The endoscopic apparatus of claim 1, wherein the laser fiber distal end is attached to the distal objective head at the distal end of the elongate scope at a location that is less distal than a most distal end of the distal objective head.

8. The endoscopic apparatus of claim 1, further comprising the elongate scope.

9. The endoscopic apparatus of claim 1, further comprising a window, separating a distal end of the laser fiber from the target calculus being drawn toward the working channel of the elongate scope via applied suction, wherein the window is transmissible to electromagnetic energy received from the laser fiber.

10. The endoscopic apparatus of claim 1, further comprising a shield, between a distal end of the laser fiber and a shielded portion of the distal objective head, the shield configured to protect the shielded portion of the distal objective head from heat generated by electromagnetic energy emitted at the distal end of the laser fiber by at least one of reflecting electromagnetic energy away from the shielded portion of the distal objective head or sinking or otherwise dissipating heat from the electromagnetic energy away from the shielded portion of the distal objective head.

11. The endoscopic apparatus of claim 1, further comprising an elongate scope sheath, including a central longitudinal sheath lumen sized and shaped to accommodate the elongate scope inserted therein, wherein the laser fiber extends proximally from the distal objective head outside the working channel of the elongate scope through the central longitudinal sheath lumen toward a proximal end of the elongate scope.

12. The endoscopic apparatus of claim 1, wherein a distal portion of the elongate scope is laterally end-user attachable to and detachable from the distal objective head in a side-by-side manner.

13. An endoscopic apparatus for use for delivering laser energy to a target within a body of a subject, the endoscopic apparatus comprising:
an elongate scope having a working channel; and
a laser fiber, including a laser fiber distal end that is attached at a distal end of the elongate scope, the laser fiber extending proximally from a distal objective head outside the working channel of the elongate scope toward a proximal end of the elongate scope, wherein the laser fiber distal end is oriented toward a location beyond the working channel of the elongate scope for delivering the laser energy toward the target, wherein the laser fiber includes one or more engagement features configured to engage with one or more corresponding mating features on the elongate scope to at least one of user-attach the laser fiber directly to the elongate scope or user-detach the laser fiber from the elongate scope.

14. An endoscopic apparatus for use for delivering laser energy to a target within a body of a subject, via an elongate scope having a working channel, the endoscopic apparatus comprising:
a laser fiber, wherein the laser fiber includes one or more engagement features configured to engage with one or more corresponding mating features on the elongate scope to at least one of user-attach the laser fiber directly to the elongate scope or user-detach the laser fiber from the elongate scope, the laser fiber including a laser fiber distal end that is user-attachable to and user-detachable from a distal end of the elongate scope, the laser fiber extending proximally from a distal objective head outside the working channel of the elongate scope toward a proximal end of the elongate scope, wherein when attached to the distal end of the elongate scope, the laser fiber distal end is oriented toward a location beyond the working channel of the elongate scope for delivering laser energy toward the target.

15. The endoscopic apparatus of claim 14, further comprising a distal objective head, sized and shaped to be coupled to a distal end of the elongate scope, wherein the laser fiber is affixed to the distal end of the elongate scope via the distal objective head.

16. The endoscopic apparatus of claim 15, wherein the distal objective head includes at least one of illumination optics, an imaging camera detector, or imaging optics.

17. The endoscopic apparatus of claim 15, further comprising a shield, between a distal end of the laser fiber and a shielded portion of the distal objective head, the shield configured to protect the shielded portion of the distal objective head from heat generated by electromagnetic energy emitted at the distal end of the laser fiber by at least one of reflecting electromagnetic energy away from the shielded portion of the distal objective head or sinking or otherwise dissipating heat from the electromagnetic energy away from the shielded portion of the distal objective head.

18. The endoscopic apparatus of claim 14, further comprising a window, separating a distal end of the laser fiber from the target, wherein the window is transmissible to electromagnetic energy received from the laser fiber.

19. The endoscopic apparatus of claim 17, further comprising an elongate scope sheath, including a central longitudinal sheath lumen sized and shaped to accommodate the elongate scope inserted therein, wherein the laser fiber extends proximally from the distal end of the elongate scope outside the working channel of the elongate scope through the central longitudinal sheath lumen toward a proximal end of the elongate scope.
